(19) United States
(12) Patent Application Publication
(10) Pub. No.: US 2023/0293418 A1
(43) Pub. Date: Sep. 21, 2023

(54) IMMEDIATE POST-PROCEDURE RECOVERY FORMULATION AND METHOD

(71) Applicant: JAMRM, LLC, Dallas, TX (US)

(72) Inventors: Alisar Zahr, Dallas, TX (US); John Koch, Fort Worth, TX (US); Tatiana Kelly, Westlake, TX (US)

(21) Appl. No.: 17/697,779

(22) Filed: Mar. 17, 2022

(51) Int. Cl. A61K 8/60; A61Q 19/00; A61K 8/9711; A61K 8/9722; A61K 8/9789; A61K 8/06

(52) U.S. Cl. CPC A61K 8/608; A61Q 19/00; A61Q 19/007; A61K 8/9711; A61K 8/9722; A61K 8/9789; A61K 8/06

(57) ABSTRACT

A skincare composition and methods for ameliorating transitory post-procedure patient discomfort and creating an environment that promotes healing after a skin rejuvenation procedure thus increasing the likelihood of patients to return for multiple treatments. The skincare composition includes an emulsion formed from active ingredients in a cosmetically acceptable carrier. The active ingredients include an effective amount of one or more neuro-cosmetic ingredients selected for increasing a concentration of beta-endorphins, reducing a concentration of CGRP, and blocking TRPV1 activity; and an effective amount of a prebiotic selected for nourishing a native microbiome of the human skin.

### BACKGROUND

### Technical Field

Novel aspects of the present disclosure relate to the field of skin care and more particularly to compositions and related methods for ameliorating pain and discomfort and creating an environment that promotes healing after a skin rejuvenation procedure that wounds skin.

### Background

Aging of the skin caused by intrinsic factors, such as genetics, and extrinsic factors, such as environmental aggressors can lead to visible changes on the skin surface including fine lines, wrinkles, pigmentation, uneven skin tone, and tactile roughness. Skin rejuvenation procedures can be used to treat these aging skin concerns. Three common types of skin rejuvenation procedures include those that wounds skin through mechanical means, through chemical aggressors, and/or through the use of energy-based devices. These common skin rejuvenation procedures vary in intensity, depth of penetration into the skin, and patient post-procedural downtime. Mechanical methods can include rejuvenation techniques with microneedling devices and/or resurfacing methods utilizing dermabrasion and dermaplaning. Chemical techniques generally involve the application of various acidic solutions to remove layers of skin. Depth of treatment depends upon factors such as acidity of the solution, types of chemicals used, and treatment time. Acids commonly used in chemical peels include alpha hydroxy acids and beta hydroxy acids. Energy-based device techniques typically employ lasers, such as carbon dioxide (CO₂), erbium (Er)-doped yttrium aluminum garnet (YAG) or radiofrequency (RF) emitters. The energy-based devices can penetrate one or more layers of skin by controlling the wavelength and amount of energy used. Each of these skin rejuvenation techniques wound the skin, which causes the body to begin a healing process that results in increased blood flow and upregulation of extracellular matrix proteins, including collagen and elastin resulting in an improvement in the health and appearance of the skin. With most procedures, the skin responds and improves after one treatment with optimal results occurring after a defined series of procedures spaced several weeks apart.

The skin rejuvenation procedures previously mentioned can be categorized based on the amount of trauma caused to the skin. Non-ablative procedures are fairly mild, treat the epidermis and do not disrupt the skin. Ablative procedures are more aggressive, disrupt the skin and can cause wounds in both the epidermis and dermis. Semi-ablative procedures, which can be performed using a hybrid fractional laser, target both the epidermis and dermis but result in less trauma than fully ablative procedures. One exemplary hybrid fractional laser combines non-ablative and ablative energy wavelengths which achieve sequential ablation of the epidermis followed by dermal coagulation to denature collagen in the dermal layer.

These skin rejuvenation procedures require multiple treatments over a period of time to achieve full benefit and satisfaction. However, these procedures collectively lead to patient discomfort by the nature of their mechanism of action. These procedures can leave the patient with their skin in a state of irritation and pain. The skin appears very red due erythema, and it is warm to the touch and itchy. Even though the results are notable, the negative experience of these procedures reduces the likelihood that the patient will return to complete the optimal series of treatments.

### SUMMARY OF THE INVENTION

Novel aspects of the present disclosure are directed to a method for ameliorating discomfort and creating an environment that promotes healing after a skincare rejuvenation procedure that wounds the skin. The method includes the steps of increasing a concentration of beta-endorphins in the skin to reduce transmission of pain signals; reducing a concentration of calcitonin gene-related peptide (CGRP) to reduce pain perception; blocking transient receptor potential cation channel subfamily V member (TRPV1) activity to decrease nociceptor sensitivity; and nourishing a native microbiome of the human skin to promote skin barrier repair.

Novel aspects of the present disclosure are also directed to a skincare composition for ameliorating discomfort and creating an environment that promotes healing after a skincare rejuvenation procedure that wounds the skin. The skincare composition is formed from active ingredients mixed with a cosmetically acceptable carrier. The active ingredients include an effective amount of one or more neuro-cosmetic ingredients selected for increasing a concentration of beta-endorphins to reduce transmission of pain signals, reducing a concentration of CGRP to reduce pain perception, and blocking TRPV1 activity to decrease nociceptor sensitivity; and an effective amount of a prebiotic selected for nourishing a native microbiome of the human skin to promote skin barrier repair.

Novel aspects of the present disclosure are also directed to a method of rejuvenating skin. The method includes the steps of performing a skin rejuvenation procedure on skin, wherein the skin rejuvenation procedure wounds the skin; and providing a skincare composition to ameliorate discomfort and create an environment that promotes healing after the skincare rejuvenation procedure. The skincare composition can be an emulsion formed from active ingredients in a cosmetically acceptable carrier, the active ingredients including an effective amount of one or more neuro-cosmetic ingredients selected for increasing a concentration of beta-endorphins, reducing a concentration of CGRP, and blocking TRPV1 activity; an effective amount of a prebiotic selected for nourishing a native microbiome of the human skin; an effective amount of a moisturizer; and an effective amount of an antioxidant.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying figures, wherein:

FIGS. 1A and 1B are charts depicting an exemplary wound healing process experienced after a conventional skin rejuvenation procedure utilizing lasers and microneedling, respectively;

FIG. 2 is a flowchart of a method for ameliorating discomfort after a skin rejuvenation procedure that wounds skin in accordance with an illustrative embodiment; and

FIG. 3 is a flowchart of a method for rejuvenating skin according to an illustrative embodiment.

### DETAILED DESCRIPTION

The more invasive skin rejuvenation procedures typically provide the most obvious benefit, particularly when repeated over multiple treatment sessions. However, these skin rejuvenation procedures can cause significant levels of pain and discomfort, usually in the form of irritation, heat sensation, redness, and itchiness. This pain and discomfort can dissuade patients from returning for additional treatment, which means less than optimal results for the patient. Conventional post-treatment skincare formulations are moisturizers with an optional sunscreen, which cannot adequately ameliorate the painful side-effects of treatment. Other post-treatment skincare formulations are complex and costly regimens that require application of multiple different post-treatment products to address different post-procedure symptoms. For example, at least one conventional post-procedure skincare regimen requires four separate products: a facial cleanser, a repair complex that includes a growth factor ingredient to stimulate collagen formation, a hydration cream, and a moisturizer. Compliance with this post-procedure skincare regimen results in the delayed introduction of soothing ingredients, which exposes the patient to a longer period of discomfort and at a time when healing of the skin barrier is well underway. A number of other conventional skincare compositions for application after skin rejuvenation procedures are anhydrous and/or occlusive and focus on moisturizing the skin but not creating an environment that promotes optimal healing and alleviates patient discomfort.

To the Applicant's knowledge, none of the prior art discloses a post-procedure product that has unique neuro-cosmetic ingredients to soothe the skin and promote skin rejuvenation, and which can be applied at least within 15 minutes of the skin rejuvenation procedure, or more particularly within 10 minutes of the skin rejuvenation procedure. Likewise, Applicant is unaware of a post-procedure product that has unique neuro-cosmetic ingredients to soothe the skin and promote skin rejuvenation, and which can be applied at least within 5 minutes of the skin rejuvenation procedure, or more particularly substantially immediately after the skin rejuvenation procedure. Whereas prior art does not contain disclosure on the delivery of neuro-cosmetic ingredients, this present invention discloses a skincare formulation which delivers unique neuro-calming ingredients to alleviate patient discomfort at least within 15 minutes, or more particularly, within 10 minutes of the skin rejuvenation procedure.

Novel aspects of this disclosure recognize the need for a skincare formulation that can be applied to human skin as a post-procedure skincare formulation that can soothe the various uncomfortable side effects of skin rejuvenation procedures with a single product using select ingredients, and by stimulating the body's natural biological response to injury. Importantly, the novel skincare formulation can be applied while the skin barrier is compromised, in some cases substantially, immediately after the skin rejuvenation procedure, which promotes optimal delivery and uptake of neuro-cosmetics that target certain biological responses to reduce pain perception, prebiotics that promote a balanced microbiome, and optional moisturizers that soften the skin and antioxidants for reducing the presence of free radicals. The skincare formulation also lacks topical anesthetics and solvent-type alcohols, which can be associated with irritation and toxicity complications. Thus, an environment that promotes healing is created by inclusion of certain ingredients that promote the balanced microbiome, moisturize the skin, reduce free radicals. Additionally, the environment that promotes healing also excludes ingredients that are believed to be hinder healing, e.g., anesthetics, solve-type alcohols, and an occlusive carrier.

Ablative skincare rejuvenation procedures disrupt the skin and cause wounds that penetrate the epidermis and dermis, which stimulates collagen production in the deeper layers of skin. Accordingly, ablative skincare procedures are ideal for treating deeper wrinkles, discoloration, textural imperfections, and deeper acne scars. Exemplary devices that can be used for providing ablative skincare procedures include a CO₂, Erbium, Erbium/YAG lasers.

Non-ablative rejuvenation skincare procedures also stimulate the production of collagen but create injury to the dermal layers without compromising the epidermis. These non-ablative skincare procedures are ideal for treating fine lines, mild scarring, light discoloration, and superficial sun damage. Exemplary devices that can be used to provide non-ablative skincare procedures include non-ablative fractional lasers and non-ablative RF transmitters to cause denaturation and acute inflammation of the dermal layer.

A semi-ablative rejuvenation skincare procedure, which results in some ablation of the epidermal layer and coagulation of the dermal layer, can also stimulate collagen production in deeper layers of skin but without the lengthy recovery period required by ablative skincare procedures. In some embodiments, the semi-ablative skincare procedure is provided by a hybrid fractional laser, which is an energy-based device that includes ablative and non-ablative energy wavelengths working in concert that results in a sequential ablation of the epidermis followed by dermal coagulation (i.e., dermal collagen denaturing).

While the skin rejuvenation procedures described above were caused by energy-based devices, in other embodiments the skin rejuvenation procedures can be a procedure that includes singularly or a combination of mechanical rejuvenation and chemical resurfacing. Examples of mechanical rejuvenation procedures include dermaplaning, dermabrasion and microneedling. Chemical resurfacing includes acidic peels.

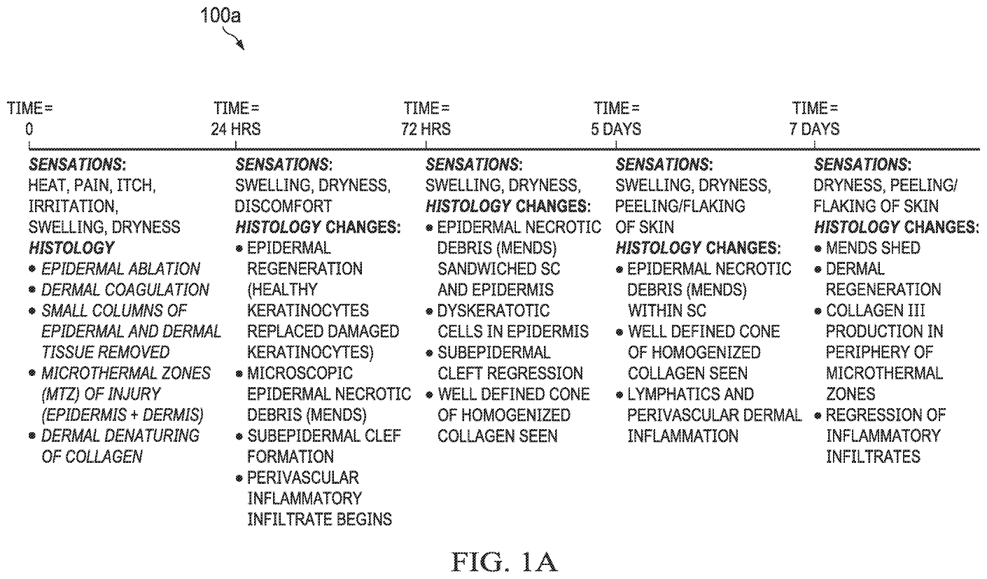

FIG. 1A is a chart depicting an exemplary wound healing process experienced by a patient after a conventional skin rejuvenation procedure. In particular, the chart 100a depicts the various symptoms experienced by the patient and observable histological changes that occur throughout a 7-day recovery period after a laser-based rejuvenation procedure. In some cases, symptoms and length of recovery can differ based on a degree of injury and quality of skin, as well as the patient's age.

The skin rejuvenation procedure ends at time=0, and within the first 24 hours the patient experiences sensations of heat, pain, itchiness, irritation, swelling, and dryness. Histological changes include epidermal ablation and dermal coagulation with dermal denaturing of collagen. In addition, small columns of epidermal and dermal tissue are removed, and microthermal zones (MTZs) of injury are observed in the epidermis and dermis.

Between 24 and 72 hours after the skin rejuvenation procedure, the patient experiences sensations of swelling, dryness, and discomfort. Histologically, the patient experiences epidermal regeneration characterized by replacement of damaged keratinocytes by healthy keratinocytes. Microscopic epidermal necrotic debris (MENDS) is observed in the epidermis and subepidermal clef formation occurs. Perivascular inflammatory infiltrate begins.

Between 72 hours and 5 days, the patient experiences sensations of swelling and dryness. Histologically, the MENDS have migrated from the epidermis and can be found between the epidermis and the stratum corneum. Dyskeratotic cells are present in the epidermis and subepidermal cleft regression occurs. A well-defined cone of homogenized collagen is observed.

Between 5 days and 7 days, the patient experiences sensations of swelling and dryness, along with peeling, flaking skin. Histologically, MENDS is observed within the stratum corneum, and lymphatics and perivascular dermal inflammation is observed. The well-defined cone of homogenized collagen is maintained.

After 7 days, the patient continues experiencing sensations of dryness and peeling, flaking skin. Histologically, the MENDS are shed and dermal regeneration begins. Collagen III production in the periphery of the MTZs is observed, along with the regression of inflammatory infiltrates.

FIG. 1B is a chart depicting an exemplary wound healing process experienced by a patient after another conventional skin rejuvenation procedure. In particular, the chart 100b depicts the various symptoms experienced by the patient and observable histological changes that occur throughout an 8-month recovery period after a microneedling procedure. In some cases, symptoms and length of recovery can differ based on a degree of injury and quality of skin, as well as the patient's age.

The skin rejuvenation procedure ends at time=0, and within the first 2-8 hours the patient experiences sensations of pain, swelling, heat, and redness. Histologically, the skin can be seen to be in the inflammatory phase. Focal damage to the superficial blood vessels and collagen bundles is observed along with the release of platelets and neutrophils.

In the time period between 2-8 hours and up to 5 days, the patient continues to experience sensations of swelling, redness, pain, and heat. Histologically, rapid healing of microchannels is observed. In addition, transdermal migration of keratinocytes occurs.

The next time period between 5 days and up to 7 days, the patient continues to experience swelling and redness. Histological samples shows evidence of the inflammatory phase in the skin.

Between 7 days and 8 months, the patient no longer experiences any sensations attributed to the rejuvenation process, but histological changes continue. In particular, monocytes/macrophage infiltration is observed. Platelet growth factors are released, and keratinocytes reestablish the basement membrane (i.e., laminin and collagen). Production of fibroblast, fibronectin, and collagen scaffold are also observed.

After 8 months, histological changes include remolding of the skin, epidermal thickening, and neoformation of Collagen I, III, and IV.

The wound healing continuums depicted in FIGS. 1A and 1B are exemplary and not meant to be limiting. The sensations and the histological changes are generally similar regardless of the type of skin rejuvenation procedure i.e., ablative, non-ablative, or semi-ablative (whether by hybrid fractional lasers or otherwise) but the actual length of recovery and the severity of the sensations vary depending upon the degree of injury to the skin, age, and state of health of the patient.

The skincare formulation described herein is formed from a plurality of active ingredients mixed with a cosmetically acceptable carrier. The cosmetically acceptable carrier can be a non-occlusive or semi-occlusive lotion or cream. In a particular embodiment, the cosmetically acceptable carrier is a semi-occlusive lotion or cream, which helps to reduce moisture loss like an occlusive-type carrier, but which allows the skin to breathe. Breathability of the carrier is a factor that improves the environment of the skin by reducing the likelihood of infection and promoting healing. Restated, the cosmetically acceptable carrier is not an occlusive lotion or cream, which prevents breathability of the skin. Non-limiting examples of the cosmetically acceptable carrier can include a traditional non-occlusive oil-in-water emulsion, a water-in-oil emulsion, a water-in-silicone emulsion, or an oil-in water emulsion paired with a film former. The carrier base can form about 65-99.6 wt % of the formulation. Non-limiting examples of a film former include synthetic or natural resins, copolymers of acrylic acid or acrylates, chitosan and its derivatives, polymers of cellulose, polyesters, polyurethanes, copolymers of starch, copolymers of styrene, polymers of VA and/or VP, and/or waxes. More specific examples of film former can include C12-22 alkyl methacrylate copolymer, ethyl cellulose, butyl ester of PVM/MA copolymer, VP/eicosene copolymer, trimethylsiloxysilicate, and adipic acid/neopentyl glycol/trimellitic anhydride copolymer.

The active ingredients include one or more neuro-cosmetic ingredients. Neuro-cosmetic ingredients are bioactive ingredients that can be topically applied, and which can affect innervation sites of human skin by penetration of the skin barrier and/or through signaling mechanisms. The neuro-cosmetic ingredients in the exemplary skincare formulation described herein are selected to reduce inflammation, itchiness, pain, and neuro-sensitivity through three major modes of action: by increasing the concentration of beta-endorphins, reducing concentration of CGRP and blocking the activity of TRPV1. Blocking TRPV1 activity can include blocking TRPV1 activation or by blocking TRPV1 reception.

Beta-endorphins are peptides that are agonists of the opioid receptor in the human body. Beta-endorphins are implicated in nociception, i.e., pain perception, of the central nervous system and the peripheral nervous system. Binding of beta-endorphins to opioid receptors reduces the release of neurotransmitters responsible for transmission of pain signals. Additionally, beta-endorphins inhibit the release of the GABA neurotransmitter, which is responsible for inhibiting the release of dopamine. Thus, the increased concentration of beta-endorphins results in the greater release of dopamine, which increases feelings of relaxation and well-being.

Beta-endorphins are produced by keratinocytes and sensory neurons. Increased production of beta-endorphins in the skin can be achieved by including one or more of the following topical active ingredients in a skincare treatment: Helichrysum italicum extract, Daucus carota sativa root extract, Lavandula stoechas extract, Crithmum maritimum extract, and acetyl dipeptide-1 cetyl ester.

TRPV1 is a polymodal nociceptor, i.e., pain receptor, that can be found on the neurons innervating the skin, and in keratinocytes. Irritation and injury to the skin, such as the kind caused by skin rejuvenation procedures, causes the release inflammatory mediators that increase the sensitivity of TRPV1 and other nociceptors to stimuli, which manifests as an increased sensitivity to pain, itchiness, discomfort, and premature aging. CGRP is an amino acid peptide that is produced by human neurons when nerves are activated by specific agonists of TRPV1 during injury. Thus, CGRP is believed to be an inflammatory mediator associated with pain perception.

Targeting TRPV1 with an antagonist reduces sensations of pain attributed to inflammatory and neuropathic pain. A corresponding reduction in the concentration of CGRP can also achieved by targeting TRPV1. TRPV1 activity can be reduced by competitive antagonists or non-competitive antagonists. These antagonists can be provided by one or more of the following active ingredients: Albatrellus confluens extract, which blocks the TRPV1 receptor, and Opuntia ficus-indica callus culture extract, which blocks TRPV1 activation.

Thus, one or more neurocosmetic ingredients include one or more of Helichrysum angustifolium flower, Albatrellus confluens extract, Daucus carota sativa root extract, Opuntia ficus-indica callus culture extract, acetyl dipeptide-1 cetyl ester. In one embodiment, the one or more neurocosmetic ingredients is present in the cosmetic formulation in an effective amount. As used herein, the term “effective amount” means a sufficient amount of a compound that can significantly induce a positive modification the condition being treated, but low enough to avoid unwanted side effects, within the scope of sound judgment of a skilled artisan. The effective amount of a compound may vary with the particular condition being treated, the age and condition of the biological subject being treated, the severity of the condition, the duration of the treatment, and other factors within the knowledge and expertise of the skilled artisan.

The active ingredients of the skincare formulation also include an effective amount of a prebiotic that can nourish the native microbiome of the human skin to promote healthy skin. The human skin hosts a range of microscopic organisms, i.e., good bacteria, that form the microbiome. Presence of the good bacteria on the skin prevents proliferation of bad bacteria on the skin. Unhealthy skin due to an unbalanced microbiome results in a compromised barrier and increased sensitivity. Non-limiting examples of prebiotics include plant fibers such as beta-glucan and inulin, Laminaria digitata extract, Chlorella vulgaris extract, and Manihot esculenta extract.

The active ingredients of the skincare formulation also include an effective amount of a moisturizer. The moisturizer can be lipophilic emollients, and/or humectants. Examples of the moisturizer include at least one of glycerin, saccharide isomerate, sorbitol, sodium hyaluronate, jojoba esters, squalene, cholesterol, natural vegetable oils and their derivatives, phytosphingosine, ceramides, Citrullus lanatus fruit extract, Lens esculenta fruit extract, Pyrus malus fruit extract, Saccharomyces cerevisiae extract and Bacillus ferment. The Bacillus ferment can be obtained from a marine-based microorganism.

The active ingredients of the skincare formulation also include an effective amount of an antioxidant. Non-limiting examples of the antioxidant can include one or more of plant polyphenols including flavonoids and phenolic acids, carotenoids, ubiquinone, tocopherol or its derivatives, ascorbic acid or its derivatives including tetrahexyldecyl ascorbate, bakuchiol and acetyl zingerone.

Thus, an illustrative skincare formulation is an emulsion formed from a cosmetically acceptable carrier and active ingredients that includes an effective amount of a neuro-cosmetic ingredients and an effective amount of one or more prebiotics. In another embodiment, the active ingredients can also include effective amounts of one or more moisturizers and one or more antioxidants.

In a particular embodiment, the effective amount of one or more neuro-cosmetic ingredients for ameliorating pain and discomfort after a semi-ablative skin rejuvenation procedure is between 0.0526-2.629 wt % of the skincare formulation, or more specifically between 0.213-1.893 wt % of the skincare formulation. In a particular embodiment, the effective amount of a neuro-cosmetic ingredient for ameliorating pain and discomfort after a semi-ablative skin rejuvenation procedure is about 1.05 wt % of the skincare formulation.

In a more specific embodiment, the one or more neuro-cosmetic ingredients includes an effective amount of one or more ingredients selected for increasing a concentration of beta-endorphins. The effective amount of the one or more ingredients selected for increasing the concentration of beta-endorphins can be between 0.0158-0.791 wt % of the skincare formulation, or more specifically between 0.063-0.570 wt % of the skincare formulation. In this more specific embodiment, the one or more neuro-cosmetic ingredients includes an effective amount of one or more ingredients selected for blocking TRPV1 receptors. The effective amount of the one or more ingredients selected for blocking TRPV1 receptors can be between 0.037-1.838 wt % of the skincare formulation, or more specifically between 0.147-1.323 wt % of the skincare formulation.

One or more of the active ingredients can have secondary mechanisms of action that are beneficial for incorporation into a skincare composition for ameliorating discomfort after a skin rejuvenation procedure. For example, Opuntia ficus-indica callus culture extract and Saccharomyces cerevisiae extract also downregulates matrix metalloproteinase-1 (MMP1), an enzyme responsible for the breakdown of extracellular matrix, specifically interstitial collages of types I, II, and III. Daucus carota sativa extract inhibits release of CGRP and also protects skin from erythema (redness). Albatrellus confluens extract can reduce discomfort and irritation, as well as protect from heat stress. Acetyl dipeptide-1 cetyl ester can also reduce thermal sensitivity. Helichrysum italicum extract can counteract the effect of cortisol and reinforce the skin barrier by improving the production of adhesion proteins.

Conventional formulations for application after skin rejuvenation procedures include cytotoxic ingredients that are detrimental to cells, such as solvent-type alcohols including ethanol or isopropyl alcohol. The alcohol removes lipids and moisturizing factors from the skin, which results in a lengthier wound healing process. These conventional formulations also often include topical anesthetics, such as benzocaine, for reducing sensations of pain and irritation. However, the use of topical anesthetics is believed to increase the risk of developing local anesthetic system toxicity (LAST). Additionally, topical anesthetics such as benzocaine, lidocaine, and tetracaine only provide a temporary numbing effect by a mechanism of action targeting the sodium ion channels on the internal surface of nerve cell membranes. However, once the numbing effect abates, known irritation reactions can manifest. These side effects, such as itchiness, redness, and scaling of the skin, may result in enough discomfort to dissuade patients from returning for additional treatment. Accordingly, in some embodiments, Applicant's novel skincare formulation lacks ingredients that delay wound healing, such as alcohol and topical anesthetics.

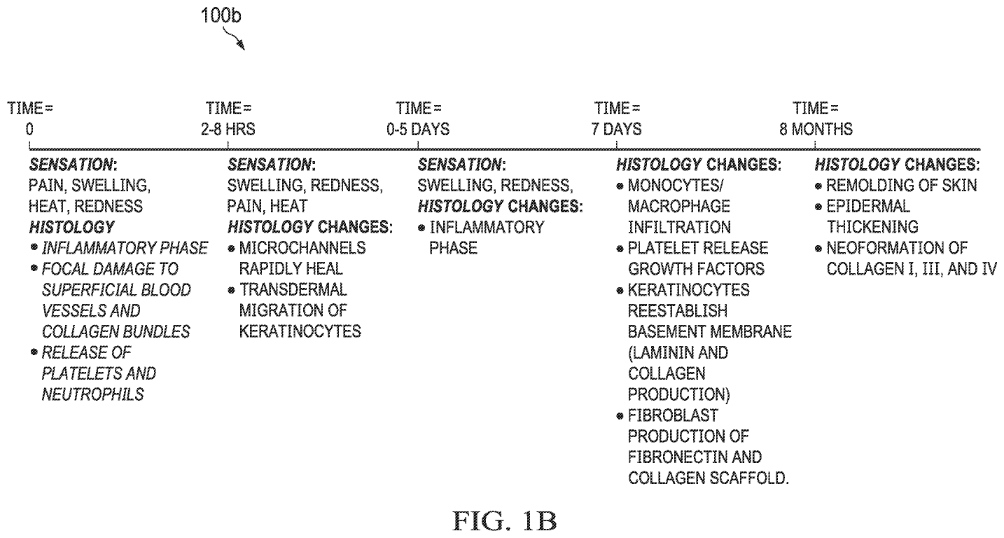

FIG. 2 is a flowchart of a process for ameliorating discomfort after a skin rejuvenation procedure that wounds skin in accordance with an illustrative embodiment. In one embodiment, the process depicted in flowchart 200 begins in response to application of a skincare formulation to skin after the skin is subjected to a rejuvenation procedure. Non-limiting examples of the rejuvenation procedure can include an ablative procedure, a non-ablative procedure, or a semi-ablative procedure, as previously described.

The process begins at step 202 by increasing a concentration of beta-endorphins in the skin. The concentration of beta-endorphins can be increased by an effective amount of one or more neuro-cosmetic ingredients, examples of which can include, but are not limited to, one or more of Helichrysum italicum extract, Albatrellus confluens extract, Daucus carota sativa root extract, Opuntia ficus-indica callus culture extract and acetyl dipeptide-1 cetyl ester. In Step 204, a concentration of CGRP is reduced by the effective amount of the one or more neuro-cosmetic ingredients. In a non-limiting embodiment, the concentration of CGRP is reduced by Daucus cartoa sativa root extract. In Step 206, TRPV1 activity is blocked by the effective amount of the one or more neuro-cosmetic ingredients. TRPV1 activity can be blocked by blocking TRPV1 activation or by blocking TRPV1 reception. TRPV1 activation can be blocked by an effective amount of Opuntia ficus-indica callus culture extract. TRPV1 reception can be blocked by an effective amount of Albatrellus confluens extract. In Step 208, the native microbiome of the human skin is nourished. The native microbiome can be nourished by an effective amount of a prebiotic, an example of which can include inulin.

In one or more embodiments, the process depicted in flowchart 200 can include an additional step of increasing a moisture content of the skin with an effective amount of a moisturizer to promote epidermal barrier repair. The moisturizer can be lipophilic emollients and/or humectants, examples of which can include glycerin, saccharide isomerate, sorbitol, sodium hyaluronate, jojoba esters, squalene, cholesterol, natural vegetable oils and their derivatives, phytosphingosine, ceramides, Citrullus lanatus fruit extract, Lens esculenta fruit extract, Pyrus malus fruit extract, Saccharomyces cerevisiae extract and Bacillus ferment.

Additionally, in one or more embodiments, the process depicted in flowchart 200 can include another step of reducing a presence of free radicals with an effective amount of an antioxidant. Non-limiting examples of the antioxidant can include one or more of plant polyphenols including flavonoids and phenolic acids, carotenoids, ubiquinone, tocopherol and tocopheryl acetate, ascorbic acid or its derivatives including tetrahexyldecyl ascorbate, bakuchiol, and acetyl zingerone.

The skincare formulation includes an effective amount of a moisturizer for increasing the moisture content of the skin; an effective amount of at least one neuro-cosmetic ingredient selected for increasing a concentration of beta-endorphins, reducing a concentration of CGRP, and blocking TRPV1 receptors; and an effective amount of a prebiotic selected for selected for nourishing the native microbiome of the human skin to promote skin barrier repair.

The skincare formulation can also include an effective amount of one or more moisturizers and an effective amount of one or more antioxidants. In some embodiments, the application step is performed within 0-48 hours of the skin rejuvenation procedure. In other embodiments, the application step is performed less than 24 hours of the skin rejuvenation procedure. In a particular embodiment, the application step is performed immediately after, i.e., 0 hours, the skin rejuvenation procedure.

Absorption of the skincare formulation into the skin induces the increasing, reducing, blocking, and nourishing steps in the skin, i.e., steps 202, 204, 206, and 208 in FIG. 2. Absorption of the skincare formulation into the skin can also cause increased hydration and reduction of a presence of free radicals when the skincare formulation includes moisturizers and antioxidants, respectively.

In one or more embodiments, the process depicted in flowchart 200 can include the initial step of applying a skincare formulation to the skin, the skincare formulation configured to ameliorate the discomfort after a skin rejuvenation procedure that wounds skin. The skincare formulation can be applied to the skin within at least 15 minutes of the skin rejuvenation procedure, or at least within 10 minutes of the skin rejuvenation procedure. In another embodiment, the skincare formulation can be applied at least within 5 minutes of the skin rejuvenation procedure, or substantially immediately after the skin rejuvenation procedure when the skin barrier is compromised. For best results, the skincare formulation can be applied three times daily for at least three days, but up to seven days for skin rejuvenating procedures with greater depths of penetration.

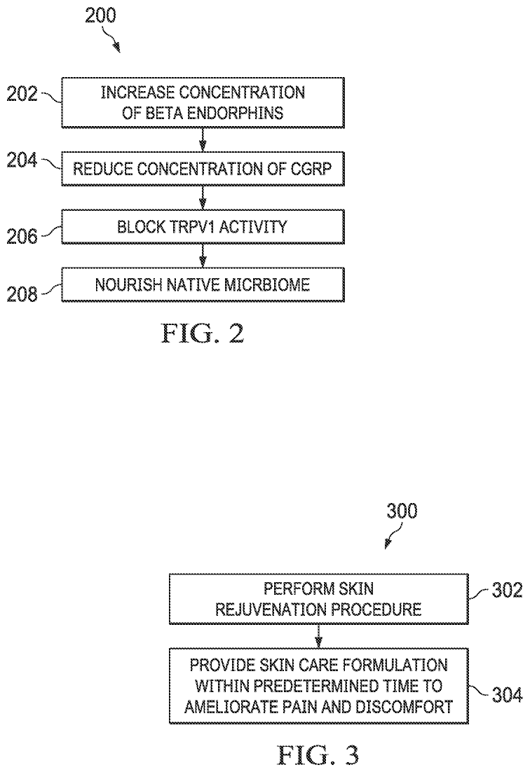

FIG. 3 is a flowchart of a method for rejuvenating skin according to an illustrative embodiment. Flowchart 300 begins at Step 302 by performing a skin rejuvenation procedure on skin. The skin rejuvenation procedure can be an ablative procedure, a non-ablative procedure, or a semi-ablative procedure. In Step 304, a skincare composition is provided for application to the skin within a predetermined period of time. The skincare composition creates an environment that promotes healing and ameliorates pain and discomfort upon application. In a non-limiting embodiment, the predetermined period of time is between 0-48 hours, and more specifically, less than about 24 hours to ameliorate discomfort after the skincare rejuvenation procedure. In a more particular embodiment, the predetermined period of time is between 0-12 hours, or between 0-6 hours. In yet another particular example, the predetermined period of time is less than 15 minutes, less than 5 minutes, or substantially immediately after the rejuvenation procedure. The skincare composition is an emulsion formed from active ingredients in a cosmetically acceptable carrier. The active ingredients can include an effective amount of a neuro-cosmetic ingredient, an effective amount of a prebiotic, and optionally effective amounts of a moisturizer and antioxidant.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. For example, the disclosure describes the application of the skincare composition described herein after a rejuvenating skincare procedure, e.g., non-ablative, semi-ablative, and ablative rejuvenation skincare procedures. However, the claimed method and skincare composition can also be applied after other forms of skin-based procedures that yield similar injuries, such as Mohs surgery and photodynamic therapy. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method for ameliorating discomfort and creating an environment that promotes healing after a skin rejuvenation procedure that wounds skin, the method comprising:
increasing a concentration of beta-endorphins in the skin to reduce transmission of pain signals;
reducing a concentration of CGRP to reduce pain perception;
blocking TRPV1 activity to decrease nociceptor sensitivity; and
nourishing a native microbiome of the human skin to promote skin barrier repair.

2. The method of claim 1, further comprising:
increasing a moisture content of the skin to promote epidermal barrier repair with an effective amount of a moisturizer, wherein the moisturizer is at least one of glycerin, saccharide isomerate, sorbitol, sodium hyaluronate, jojoba esters, squalene, cholesterol, natural vegetable oils and their derivatives, phytosphingosine, ceramides, Citrullus lanatus fruit extract, Lens esculenta fruit extract, Pyrus malus fruit extract, Saccharomyces cerevisiae extract and Bacillus ferment.

3. The method of claim 1, further comprising:
reducing a presence of free radicals with an effective amount of an antioxidant, wherein the antioxidant is at least one of plant polyphenols, carotenoids, ubiquinone, tocopherol, tocopheryl acetate, ascorbic acid, derivatives of ascorbic acid, bakuchiol, and acetyl zingerone.

4. The method of claim 2, further comprising:
applying a skincare formulation to the skin, wherein the skincare formulation includes an effective amount of a moisturizer for increasing the moisture content of the skin; an effective amount of at least one neuro-cosmetic ingredient selected for increasing a concentration of beta-endorphins, reducing a concentration of CGRP, and blocking TRPV1 receptors; and an effective amount of a prebiotic selected for nourishing the native microbiome of the human skin,
wherein absorption of the skincare formulation induces the increasing, reducing, blocking, and nourishing steps in the skin.

5. The method of claim 4, wherein the applying step is performed within at least 15 minutes after the skin rejuvenation procedure.

6. The method of claim 5, wherein the at least one neuro-cosmetic ingredient includes at least one of: Helichrysum italicum extract, Albatrellus confluens extract, Daucus cartoa sativa root extract, Opuntia ficus-indica callus culture extract or acetyl dipeptide-1 cetyl ester.

7. The method of claim 4, wherein the prebiotic comprises one or more of beta-glucan, inulin, Laminaria digitata extract, Chlorella vulgaris extract and Manihot esculenta extract.

8. The method of claim 1, wherein the skin rejuvenation procedure is one of a non-ablation procedure, semi-ablation procedure, or ablation procedure.

9. The method of claim 1, wherein the method is performed in the absence of a topical anesthetic and solvent-type alcohols.

10. The method of claim 1, wherein increasing the concentration of beta-endorphins comprises stimulating production of the beta-endorphins by keratinocytes and sensory neurons in the skin.

11. The method of claim 1, wherein the concentration of CGRP is reduced by blocking the TRPV1 receptors with an antagonist provided in a skincare formulation.

12. A skincare composition for ameliorating discomfort and creating an environment that promotes healing after a skin rejuvenation procedure, the skincare composition comprising:
active ingredients mixed with a cosmetically acceptable carrier, wherein the active ingredients include:
an effective amount of one or more neuro-cosmetic ingredients selected for increasing a concentration of beta-endorphins, reducing a concentration of CGRP, and blocking TRPV1 activity; and
an effective amount of a prebiotic selected for nourishing a native microbiome of the human skin to promote skin barrier repair.

13. The skincare composition of claim 12, wherein the active ingredients further comprise:
an effective amount of a moisturizer.

14. The skincare composition of claim 13, wherein the moisturizer includes at least one of jojoba esters, cholesterol, phytosphingosine, ceramides, Citrullus lanatus fruit extract, Lens esculenta fruit extract, and Pyrus malus fruit extract.

15. The skincare composition of claim 12, wherein the one or more neuro-cosmetic ingredients comprises an effective amount of at least one of Helichrysum italicum extract, Albatrellus confluens extract, Daucus carota sativa root extract, Opuntia ficus-indica callus culture extract or acetyl dipeptide-1 cetyl ester.

16. The skincare composition of claim 12, wherein the prebiotic comprises an effective amount of at least one of beta-glucan, inulin, Laminaria digitata extract, Chlorella vulgaris extract and Manihot esculenta extract.

17. The skincare composition of claim 12, wherein the skin rejuvenation procedure is one of a non-ablation procedure, semi-ablation procedure, or ablation procedure.

18. The skincare composition of claim 12, wherein the cosmetically acceptable carrier is one of a water-in-oil emulsion, a water-in-silicone emulsion, or an oil-in-water emulsion.

19. The skincare composition of claim 12, wherein the active ingredients further comprise:
an effective amount of an antioxidant, wherein the antioxidant is at least one of ubiquinone, tocopherol, tocopheryl acetate, tetrahexyldecyl ascorbate, and acetyl zingerone.

20. A method for rejuvenating skin, the method comprising:
performing a skin rejuvenation procedure on skin, wherein the skin rejuvenation procedure wounds the skin;
providing a skincare composition for application to the skin within 6 hours after the skincare rejuvenation procedure to ameliorate discomfort, wherein the skincare composition is an emulsion formed from active ingredients in a cosmetically acceptable carrier, and wherein the active ingredients comprise:
an effective amount of one or more neuro-cosmetic ingredients selected for increasing a concentration of beta-endorphins, reducing a concentration of CGRP, and blocking TRPV1 activity;
an effective amount of a prebiotic selected for nourishing a native microbiome of the human skin to promote skin barrier repair;
an effective amount of a moisturizer; and
an effective amount of an antioxidant.
